(19) United States
(12) Patent Application Publication
(10) Pub. No.: US 2007/0218130 A1
(43) Pub. Date: Sep. 20, 2007

(54) Bisphosphonate composition and process for the preparation thereof

(75) Inventors: Salah U. Ahmed, New City, NY (US); Pruthvipathy R. Katikaneni, Princeton, NJ (US); Gandha Naringrekar, Paramus, NJ (US); Krishna K. Venkatesh, Pomona, NY (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Barr Laboratories, Inc., Pomona, NY (US)

(21) Appl. No.: 11/546,548

(22) Filed: Oct. 12, 2006

Related U.S. Application Data
(63) Continuation of application No. 10/677,243, filed on Oct. 3, 2003, now abandoned.
(60) Provisional application No. 60/415,513, filed on Oct. 3, 2002.

(51) Int. Cl. A61K 9/20; A61K 31/66

(52) U.S. Cl. 424/465; 514/75

(57) ABSTRACT

The present invention is generally directed to pharmaceutical compositions and process for the preparation of same which are suitable for oral administration to a human patient, comprising: a pharmaceutically-acceptable salt of 4-amino-1-hydroxybutylidene-1,1-bisphosphonic acid; a non-reducing sugar diluent; a binder; a disintegrant; and a lubricant, and various other excipients.

### BACKGROUND OF THE INVENTION

1. Field of the Invention

The present invention relates generally to pharmaceutical compositions of pharmaceutically acceptable salts of 4-amino-1-hydroxy-butylidene-1,1-bisphosphonic acid and more specifically to such compositions having improved stability and potency.

2. Background Art

Oral pharmaceutical dosage forms commonly include, in addition to a pharmaceutically active ingredient, various additives or excipients such as diluents, fillers, binders, disintegrating agents, lubricants, coatings, solvents, suspending agents and dyes. The physical properties and chemical stability of any oral dosage form is, at least in part, dependent on the choice of excipient.

For example, the use of the common diluent lactose, when employed in a solid dosage form of an active ingredient having a basic nitrogen-containing functionality, can result in discoloration, chemical instability and loss of dosage strength or potency of the dosage form. The mechanism responsible for the incompatibility of lactose with basic nitrogen-containing active ingredients is believed to be due to the Maillard (or “browning”) reaction in which the basic nitrogen (typically a primary or secondary amino group) of the active ingredient reacts with the “glycosidic” hydroxyl group of lactose ultimately resulting in the formation of brown pigmented degradates. Other sugars having a “glycosidic” hydroxyl group, such as glucose, also stimulate this degredation when employed as excipients in dosage forms of basic nitrogen-containing actives. Degradation of the active ingredient in this way is particularly pronounced in the presence of water and/or elevated temperature.

The Maillard reaction is a multi-stage process that yields a number of different products. This process is diagramed below for glucose (a common reducing sugar and one of the two monosaccharides that comprise lactose) and an organic compound containing a primary amine group (“R” is used to depict the residual portion of the organic compound):

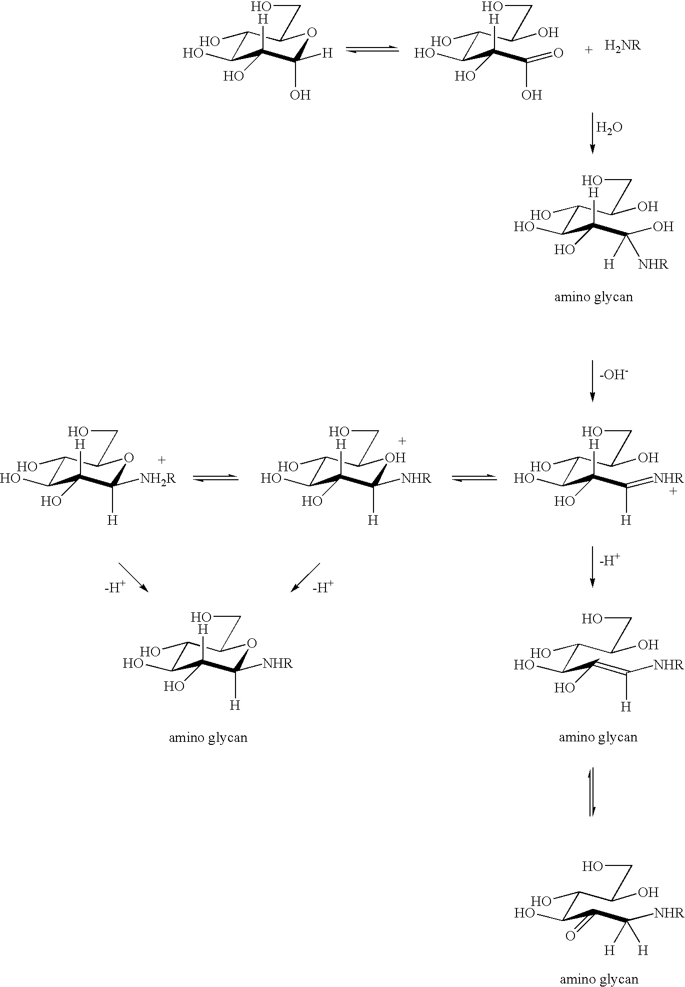

As would be expected from the above reaction scheme, the Maillard reaction is a particularly significant problem for pharmaceutical formulations that include both a reducing sugar such as lactose and an active therapeutic agent which contains an amino group. More specifically, lactose is widely used as a diluent for pharmaceutical tablet formulations due to its low price, high purity and excellent compression and stability characteristics. As shown above, however, lactose is a reducing sugar and so can react with an amino group in the active therapeutic agent of a particular pharmaceutical formulation.

A variety of bisphosphonates which bear a basic nitrogen-containing functionality have been disclosed as being useful in the treatment and prevention of diseases involving bone resorption. Representative examples may be found in the following: U.S. Pat. No. 3,962,432; U.S. Pat. No. 4,054,598; U.S. Pat. No. 4,267,108; U.S. Pat. No. 4,327,039; U.S. Pat. No. 4,621,077; U.S. Pat. No. 4,624,947; U.S. Pat. No. 4,746,654; U.S. Pat. No. 4,922,077; U.S. Pat. No. 5,994,329, U.S. Pat. No. 6,015,801 and EPO Patent Pub. No. 0,252,504.

Prior attempts to formulate bisphosphonates bearing basic nitrogen-containing functionality with a lactose diluent have focused on eliminating water in the formulation process. A dry mix process using anhydrous lactose as a diluent avoids the enhanced degradation which occurs in the presence of water.

Solid dosage forms prepared by such a process are exemplified in U.S. Pat. No. 5,882,656 which discloses a tablet formulation prepared by mixing the formulation ingredients with no hydration prior to direct compression.

The present invention solves the problem of unwanted degradation in 4-amino-1-hydroxy-butylidene-1-bisphosphonate formulations by avoiding the Maillard reaction altogether.

### BRIEF SUMMARY OF THE INVENTION

A first aspect of the present invention is directed to a pharmaceutical composition suitable for oral administration to a human, comprising:

(i) from about 0.5% to about 60% by weight of a pharmaceutically acceptable salt of 4-amino-1-hydroxybutylidene-1,1-bisphosphonic acid;

(ii) from about 10% to about 95% by weight of a non-reducing sugar selected from the group consisting of mannitol, xylitol, sorbitol, inositol, sucrose and trehalose;

(iii) from about 2% to about 60% by weight of a binder selected from the group consisting of microcrystalline cellulose, hydroxypropyl cellulose, methyl cellulose, hydroxypropyl methyl cellulose and polyvinylpyrrolidone;

(iv) from about 0.5% to about 15% by weight of a disintegrant selected from the group consisting of starch, modified starch, croscarmellose sodium, crospovidone and sodium starch glycolate; and

(v) from about 0.1% to about 7% by weight of a lubricant selected from the group consisting of calcium stearate, magnesium stearate, stearic acid, talc, hydrogenated vegetable oil and sodium stearyl fumarate.

A second aspect of the present invention is directed to a process for the preparation of a pharmaceutical composition suitable for oral administration to a human, comprising:

forming a mixture of from about 0.5% to about 60% by weight of a pharmaceutically acceptable salt of 4-amino-1-hydroxybutylidene-1,1-bisphosphonic acid, from about 10% to about 95% by weight of a non-reducing sugar selected from the group consisting of mannitol, xylitol, sorbitol, inositol, sucrose and trehalose, from about 2% to about 60% by weight of a binder selected from the group consisting of microcrystalline cellulose, hydroxypropyl cellulose, methyl cellulose, hydroxypropyl methyl cellulose and polyvinylpyrrolidone; from about 0.5% to about 15% by weight of a disintegrant selected from the group consisting of starch, modified starch, croscarmellose sodium, crospovidone and sodium starch glycolate; and from about 0.1% to about 7% by weight of a lubricant selected from the group consisting of calcium stearate, magnesium stearate, stearic acid, talc, hydrogenated vegetable oil and sodium stearyl fumarate; and

compressing said mixture into a tablet.

### BRIEF DESCRIPTION OF THE FIGURES

FIG. 1 is a flow chart illustrating a preferred process for the preparation of a tablet comprising a composition of the present invention for daily administration.

FIG. 2 is a flow chart illustrating a preferred process for the preparation of a tablet comprising another composition of the present invention for weekly or biweekly administration.

FIG. 3 is a comparative dissolution profile of a 10 mg tablet prepared with a mannitol diluent in accordance with the invention and a commercially available 10 mg tablet (reference) prepared with an anhydrous lactose diluent.

FIG. 4 is a comparative dissolution profile of a 40 mg tablet prepared with a mannitol diluent in accordance with the invention and a commercially available 40 mg tablet (reference) prepared with an anhydrous lactose diluent.

FIG. 5 is a comparative dissolution profile of a 35 mg tablet prepared with a mannitol diluent in accordance with the invention and a commercially available 35 mg tablet (reference) prepared with an anhydrous diluent.

FIG. 6 is a comparative dissolution profile of a 70 mg table prepared with a mannitol diluent in accordance with the invention and a commercial available 70 mg tablet (reference) prepared with an anhydrous diluent.

### DETAILED DESCRIPTION OF THE INVENTION

A first aspect of the present invention is directed to a pharmaceutical composition suitable for oral administration to a human, comprising:

from about 0.5% to about 60% by weight of a pharmaceutically acceptable salt of 4-amino-1-hydroxybutylidene-1,1-bisphosphonic acid;

(ii) from about 10% to about 95% by weight of a non-reducing sugar selected from the group consisting of mannitol, xylitol, sorbitol, inositol, sucrose and trehalose;

(iii) from about 2% to about 60% by weight of a binder selected from the group consisting of microcrystalline cellulose, hydroxypropyl cellulose, methyl cellulose, hydroxypropyl methyl cellulose and polyvinylpyrrolidone;

(iv) from about 0.5% to about 15% by weight of a disintegrant selected from the group consisting of starch, modified starch, croscarmellose sodium, crospovidone and sodium starch glycolate; and

(v) from about 0.1% to about 7% by weight of a lubricant selected from the group consisting of calcium stearate, magnesium stearate, stearic acid, talc, hydrogenated vegetable oil and sodium stearyl fumarate.

Examples of basic nitrogen-containing bisphosphonates which can be employed in the composition of the present invention include the pharmaceutically acceptable salts of:

4-amino-1-hydroxybutylidene-1,1-bisphosphonic acid;

N-methyl-4-amino-1-hydroxybutylidene-1,1-bisphosphonic acid;

4-(N,N-dimethylamino)-1-hydroxybutylidene-1,1-bisphosphonic acid;

3-amino-1-hydroxypropylidene-1,1-bisphosphonic acid;

3-(N,N-dimethylamino)-1-hydroxypropylidene-1,1-bisphosphonic acid;

1-hydroxy-3-(N-methyl-N-pentylamino)propylidene-1,1-bisphosphonic acid;

1-hydroxy-2-[3-pyridyl]ethylidene-1,1-bisphosphonic acid; and

4-(hydroxymethylene-1,1-bisphosphonic acid)piperidine.

Methods for the preparation of salts of 4-amino-1-hydroxy-butylidene-1,1-bisphosphonic acid and, in particular, 4-amino-1-hydroxy-butylidene-1,1-bisphosphonic acid monosodium salt trihydrate are found in U.S. Pat. No. 4,407,761 and U.S. Pat. No. 4,922,007, respectively. A method for the production of anhydrous 4-amino-1-hydroxy-butylidene-1,1-bisphosphonic acid monosodium salt is found in U.S. Pat. No. 5,849,726.

Examples of base salts of basic nitrogen-containing bisphosphonic acids include, but are not limited to, ammonium salts, alkali metal salts such as potassium and sodium (including, but not limited to, mono-, di- and tri-sodium) salts (which are preferred), alkaline earth metal salts such as calcium and magnesium salts, salts with organic bases such as dicyclohexylamine salts, N-methyl-D-glucamine, and salts with amino acids such as arginine, lysine. The non-toxic, physiologically acceptable salts are preferred. The salts may be prepared by methods known in the art, such as in U.S. Pat. No. 4,922,007, U.S. Pat. No. 5,019,651 or U.S. Pat. No. 5,908,959.

In the present invention a useful basic nitrogen-containing salt of a bisphosphonic acid is a salt of 4-amino-1-hydroxybutylidene-1,1-bisphosphonic acid, such as a sodium salt of 4-amino-1-hydroxybutylidene-1,1-bisphosphonic acid, in particular, 4-amino-1-hydroxybutylidene-1,1-bisphosphonic acid monosodium salt trihydrate or anhydrous 4-amino-1-hydroxybutylidene-1,1-bisphosphonic acid monosodium salt.

As used herein, the term “pharmaceutically effective” refers to that amount which effects the turnover of mature bone. The precise therapeutic dosage of basic nitrogen-containing bisphosphonic acid necessary to be pharmaceutically effective will vary with the age, size, sex and condition of the subject, the nature and severity of the disorder to be treated, and the like; thus, a precise pharmaceutically effective amount cannot be specified in advance and will be determined by the caregiver. However, appropriate amounts may be determined by routine experimentation with animal models. In general terms, an effective dose is about 0.01 to 1 mg/kg per day of body weight. Useful dosages include 6.53, 13.05 and 52.21 mg per day/per person of 4-amino-1-hydroxybutylidene-1,1-bisphosphonic acid monosodium salt trihydrate (equivalent to 5, 10 and 40 mg free acid equivalents) per day per person. Alternatively, the bisphosphonic acid may be administered on a once-weekly or twice-monthly basis. If administered weekly or biweekly, a useful dose of the bisphosphonic acid are 45.68 and 91.37 mg per week/per person of 4-amino-1-hydroxybutylidene-1,1-bisphosphonic acid monosodium salt trihydrate (equivalent to 35 and 70 mg free acid equivalents) per day per person.

A “non-reducing sugar diluent” as used herein refers to a sugar without a glycosidic hydroxyl group or a sugar which is otherwise incapable of reaction with the basic nitrogen of a basic nitrogen-containing compound in a Maillard-type reaction. Usefull non-reducing sugar diluents for use in the present invention are mannitol, xylitol, sorbitol, inositol, sucrose and trehalose, most preferably mannitol.

As used herein, the term “excipient” refers to the additives used to convert an active compound into a form suitable for its intended purpose. For compositions of the present invention suitable for administration to a human, the term “excipient” means those excipients described in the Handbook of Pharmaceutical Excipients, American Pharmaceutical Association, 2^{nd} Ed. (1994), which is herein incorporated by reference in its entirety. The term “excipients” is meant to include fillers, binders, disintegrating agents, lubricants, coatings, solvents, suspending agents, dyes, extenders, surfactants, auxiliaries and the like.

Binders useful in the composition and method of the present invention include, but are not limited to, hydrophilic gums such as microcrystalline cellulose, hydroxypropyl cellulose, methyl cellulose, hydroxypropyl methyl cellulose and polyvinylpyrrolidone, preferably hydroxypropyl methyl cellulose. Hydroxypropyl methyl cellulose is commercially available under the trade name “Methocel K3 Premium” from Dow Chemical Company.

Disintegrants for use in the composition and method of the present invention include, but are not limited to, one of several modified starches or modified cellulose polymers such as starch, modified starch, crosscarmallose sodium, crospovidone and sodium starch glycolate, preferably sodium starch glycolate. Sodium starch glycolate NF is commercially available under the trade name “Primojel” from Avebe.

Lubricants for use in the composition and method of the present invention include, but are not limited to, calcium stearate, magnesium stearate, stearic acid, talc, hydrogenated vegetable oil and sodium stearyl fumarate, preferably sodium stearyl fumarate. Sodium stearyl fumarate is commercially available under the trade name “Pruv” from Astra Pharmaceutical Production AB.

Substances for use as coatings in the composition and method of the present invention include, but are not limited to, hydroxypropyl methyl cellulose, hydroxypropylcellulose, titanium oxide, talc and other sweeteners, and colorants. Enteric coatings may also be employed.

The pharmaceutical composition of the present invention comprises about 0.5% to about 60% by weight of a basic nitrogen-containing bisphosphonate selected from anhydrous 4-amino-1-hydroxybutylidene-1,1-bisphosphonic acid monosodium salt and 4-amino-1-hydroxybutylidene-1,1-bisphosphonic acid monosodium salt trihydrate; about 10% to about 95% by weight of a non-reducing sugar selected from the group consisting of mannitol, xylitol, sorbitol, inositol, sucrose and trehalose; about 2% to about 60% by weight of a binder selected from the group consisting of microcrystalline cellulose, hydroxypropyl cellulose, methyl cellulose, hydroxypropyl methyl cellulose and polyvinylpyrrolidone; about 0.5% to about 15% by weight of a disintegrant selected from the group consisting of starch, modified starch, croscarmellose sodium, crospovidone and sodium starch glycolate; and about 0.1% to about 7% by weight of a lubricant selected from the group consisting of calcium stearate, magnesium stearate, stearic acid, talc, hydrogenated vegetable oil and sodium stearyl fumarate.

A preferred pharmaceutical composition of the present invention comprises about 0.5% to about 50% by weight of a basic nitrogen-containing bisphosphonate selected from anhydrous 4-amino-1-hydroxybutylidene-1,1-bisphosphonic acid monosodium salt and 4-amino-1-hydroxybutylidene-1,1-bisphosphonic acid monosodium salt trihydrate; about 20% to about 90% by weight of a non-reducing sugar selected from the group consisting of mannitol, xylitol, sorbitol, inositol, sucrose and trehalose; about 5% to about 50% by weight of a binder selected from the group consisting of microcrystalline cellulose, hydroxypropyl cellulose, methyl cellulose, hydroxypropyl methyl cellulose and polyvinylpyrrolidone; about 0.5% to about 10% by weight of a disintegrant selected from the group consisting of starch, modified starch, croscarmellose sodium, crospovidone and sodium starch glycolate; and about 0.25% to about 5% by weight of a lubricant selected from the group consisting of calcium stearate, magnesium stearate, stearic acid, talc, hydrogenated vegetable oil and sodium stearyl fumarate.

A more preferred pharmaceutical composition of the present invention comprises about 1% to about 30% by weight of a basic nitrogen-containing bisphosphonate selected from anhydrous 4-amino-1-hydroxybutylidene-1,1-bisphosphonic acid monosodium salt and 4-amino-1-hydroxybutylidene-1,1-bisphosphonic acid monosodium salt trihydrate; about 30% to about 80% by weight of a non-reducing sugar selected from the group consisting of mannitol, xylitol, sorbitol, inositol, sucrose and trehalose; about 10% to about 45% by weight of a binder selected from the group consisting of microcrystalline cellulose, hydroxypropyl cellulose, methyl cellulose, hydroxypropyl methyl cellulose and polyvinylpyrrolidone; about 0.5% to about 8% by weight of a disintegrant selected from the group consisting of starch, modified starch, croscarmellose sodium, crospovidone and sodium starch glycolate; and about 0.5% to about 3% by weight of a lubricant selected from the group consisting of calcium stearate, magnesium stearate, stearic acid, talc, hydrogenated vegetable oil and sodium stearyl fumarate.

One useful pharmaceutical composition comprises about 0.5 to 60% by weight of 4-amino-1-hydroxybutylidene-1,1-bisphosphonate as an active ingredient; about 30 to 95% by weight of mannitol; about 20 to 40% by weight of hydroxypropyl methyl cellulose; about 1 to 15% by weight of sodium starch glycolate; and about 0.25 to 7% by weight of sodium stearyl fumarate.

Another useful pharmaceutical composition in accordance with the present invention comprises: about 0.5 to 50% by weight of 4-amino-1-hydroxybutylidene-1,1-bisphosphonate; about 40 to 90% by weight of mannitol; about 5 to 30% by weight of hydroxypropyl methyl cellulose; about 1 to 10% by weight of sodium starch glycolate; and about 0.5 to 5% by weight of sodium stearyl fumarate.

Yet another useful pharmaceutical composition of the present invention comprises about 1% to about 30% by weight of 4-amino-1-hydroxybutylidene-1,1-bisphosphonate; about 50% to about 80% by weight of mannitol; about 10% to about 20% by weight of hydroxypropyl methyl cellulose; about 2% to about 8% by weight of sodium starch glycolate; and about 1% to about 3% by weight of sodium stearyl fumarate.

The pharmaceutical compositions are generally in the form of tablets. The tablets are, for example, from 50 mg to 1.0 g in net weight, such as from 100 to 500 mg net weight, in particular 200 to 300 mg net weight.

A useful pharmaceutical composition for weekly or biweekly administration comprises about 10 to 60% by weight of a basic nitrogen-containing bisphosphonate as an active ingredient; about 10 to 50% by weight of mannitol; about 20 to 60% by weight of microcrystalline cellulose; about 0.5 to 10% by weight of croscarmellose sodium; and about 0.1 to 3% by weight of magnesium stearate.

Another useful pharmaceutical composition for weekly or biweekly administration, in accordance with the present invention comprises: about 15 to 40% by weight of a pharmaceutically acceptable salt of 4-amino-1-hydroxybutylidene-1,1-bisphosphonic acid; about 20 to 40% by weight of mannitol; about 30 to 50% by weight of microcrystalline cellulose; about 0.5 to 5% by weight of croscarmellose sodium; and about 0.25 to 2% by weight of sodium stearyl fumarate.

Yet another useful pharmaceutical composition for weekly or biweekly administration, in accordance with the present invention, comprises about 20% to about 30% by weight of a pharmaceutically acceptable salt of 4-amino-1-hydroxybutylidene-1,1-bisphosphonic acid; about 25% to about 35% by weight of mannitol; about 35% to about 45% by weight of microcrystalline cellulose; about 0.5% to about 1.5% by weight of croscarmellose sodium; and about 0.5% to about 1% by weight of magnesium stearate.

The pharmaceutical compositions for weekly or biweekly administration are generally in the form of tablets. The tablets are, for example, from 50 mg to 1.0 g in net weight, such as 100 to 500 mg net weight and 250 to 400 mg net weight.

Non-limiting examples of pharmaceutical compositions for commercial application are as follows (by weight percent):

Tablets of 5 mg strength:
about 3.3% 4-amino-1-hydroxybutylidene-1,1-bisphosphonic acid monosodium salt trihydrate; about 75.7% mannitol; about 13% hydroxypropyl methyl cellulose; about 6% sodium starch glycolate; and about 2% sodium stearyl fumarate.

Tablets of 10 mg strength:
about 6.5% 4-amino-1-hydroxybutylidene-1,1-bisphosphonic acid monosodium salt trihydrate; about 72.5% mannitol; about 13.5% hydroxypropyl methyl cellulose; about 5.5% sodium starch glycolate; and

about 2% sodium stearyl fumarate.

Tablets of 40 mg strength:
about 26.1% 4-amino-1-hydroxybutylidene-1,1-bisphosphonic acid monosodium salt trihydrate; about 52.9% mannitol; about 15% hydroxypropyl methyl cellulose; about 4% sodium starch glycolate; and about 2% sodium stearyl fumarate.

Tablets of 35 mg strength:
about 26.1% 4-amino-1-hydroxybutylidene-1,1-bisphosphonic acid monosodium salt trihydrate; about 32.3% mannitol; about 40% microcrystalline cellulose; about 1% croscarmellose sodium; and about 0.6% magnesium stearate.

Tablets of 70 mg strength:
about 26.1% 4-amino-1-hydroxybutylidene-1,1-bisphosphonic acid monosodium salt trihydrate; about 32.3% mannitol; about 40% microcrystalline cellulose; about 1% croscarmellose sodium; and about 0.6% magnesium stearate.

The pharmaceutical tablet compositions of the present invention may contain one or more additional formulation ingredients selected from a wide variety of excipients known in the pharmaceutical formulation art. According to the desired properties of the tablet, any number of ingredients may be selected, alone or in combination, based upon their known uses in preparing tablet compositions.

The disclosed compositions may be prepared as solid dosage forms, preferably as tablets, for medical administration.

The term “tablet” as used herein is intended to encompass compressed pharmaceutical dosage formulations of all shapes and sizes, whether coated or uncoated.

The following table is a tabular representation of the above description.

| | % of the tablet's weight | | | | |
|---|---|---|---|---|---|
| | | | | More | |
| Ingredient | # | Generally | Preferable | Preferable | Actual |
| Alendronate | A | 10-60 | 15-40 | 20-30 | 26 |
| | B | 0.5-60 | 0.5-50 | 1-30 | 3-26 |
| | * | 0.5-60 | 0.5-50 | 1-30 | |
| Mannitol | A | 10-50 | 20-40 | 25-35 | 32 |
| | B | 30-95 | 40-90 | 50-80 | 53-75 |
| | * | 10-95 | 20-90 | 30-80 | |
| Microcrystalline | A | 20-60 | 30-50 | 35-45 | 40 |
| Cellulose(A) | B | 2-40 | 5-30 | 10-20 | 13-15 |
| Hydroxypropyl | * | 2-60 | 5-50 | 10-45 | |
| methyl | | | | | |
| Cellulose(B) | | | | | |
| Croscarmellose | A | 0.5-10 | 0.5-5 | 0.5-1.5 | 1 |
| Sodium(A) | B | 1-15 | 1-10 | 2-8 | 4-6 |
| Sodium Starch | * | 0.5-15 | 0.5-10 | 0.5-8 | |
| Glycolate(B) | | | | | |
| Magnesium | A | 0.1-3 | 0.25-2 | 0.5-1 | 0.6 |
| Stearate(A) | | | | | |
| Sodium Stearyl | B | 0.25-7 | 0.5-5 | 1-3 | 2 |
| fumarate(B) | * | 0.1-7 | 0.25-5 | 0.5-3 | |

Legend:
A - Formulation for the 35 and 70 mg strengths
B - Formulation for the 10, 20, and 40 mg strengths
* Proposed limits from the values used in all the strengths

A second aspect of the present invention is directed to A second aspect of the present invention is directed to a process for the preparation of a pharmaceutical composition suitable for oral administration to a human, comprising:

forming a mixture of from about 0.5% to about 60% by weight of a pharmaceutically acceptable salt of 4-amino-1-hydroxybutylidene-1,1-bisphosphonic acid, from about 10% to about 95% by weight of a non-reducing sugar selected from the group consisting of mannitol, xylitol, sorbitol, inositol, sucrose and trehalose, from about 2% to about 60% by weight of a binder selected from the group consisting of microcrystalline cellulose, hydroxypropyl cellulose, methyl cellulose, hydroxypropyl methyl cellulose and polyvinylpyrrolidone; from about 0.5% to about 15% by weight of a disintegrant selected from the group consisting of starch, modified starch, croscarmellose sodium, crospovidone and sodium starch glycolate; and from about 0.1% to about 7% by weight of a lubricant selected from the group consisting of calcium stearate, magnesium stearate, stearic acid, talc, hydrogenated vegetable oil and sodium stearyl fumarate; and

compressing said mixture into a tablet.

The following examples of processing conditions and parameters are given for the purpose of illustrating the present invention and shall not be construed as being limitations on the scope or spirit of the invention.

### EXAMPLES

### Examples 1-3

### Preparation of Tablets of 4-Amino-1-hydroxybutylidene-1,1-bisphosphonic acid monosodium salt trihydrate

| | | | Example 3 |
|---|---|---|---|
| | Example 1 | Example 2 | 40 mg |
| | 5 mg Potency | 10 mg Potency | Potency |
| | Per Tablet | Per Tablet | Per Tablet |
| Ingredients | (mg) | (mg) | (mg) |
| 4-amino-1- | 6.53 | 13.05 | 52.21 |
| hydroxybutylidene-1,1- | | | |
| bisphosphonic acid | | | |
| monosodium salt | | | |
| trihydrate | | | |
| Mannitol, USP | 151.47 | 144.95 | 105.79 |
| (Pearlitol 200 SD) | | | |
| HPMC 2208, USP | 26 | 27 | 30 |
| (Methocel K3 | | | |
| Premium) | | | |
| Sodium starch | 12 | 11 | 8 |
| glycolate, NF | | | |
| (Primojel) | | | |
| Sodium stearyl | 4 | 4 | 4 |
| fumarate (Pruv) | | | |
| Tablet weight | 200 | 200 | 200 |

The following procedure was followed for each of the above tablet formulations:

a. mannitol, 4-amino-1-hydroxybutylidene-1,1-bisphosphonic acid monosodium trihydrate and hydroxypropyl methyl cellulose were deagglomerated by passing through a screen;

b. the deagglomerated components from step (a) were mixed in a high shear mixer granulator for three minutes;

c. deagglomerated sodium starch glycolate was added and the mixture subjected to the action of a high shear mixer granulator for two minutes;

d. sodium stearyl fumarate was added and the mixture subjected to the action of a high shear mixer granulator for one minute; and

e. the final mixture was compressed into tablets.

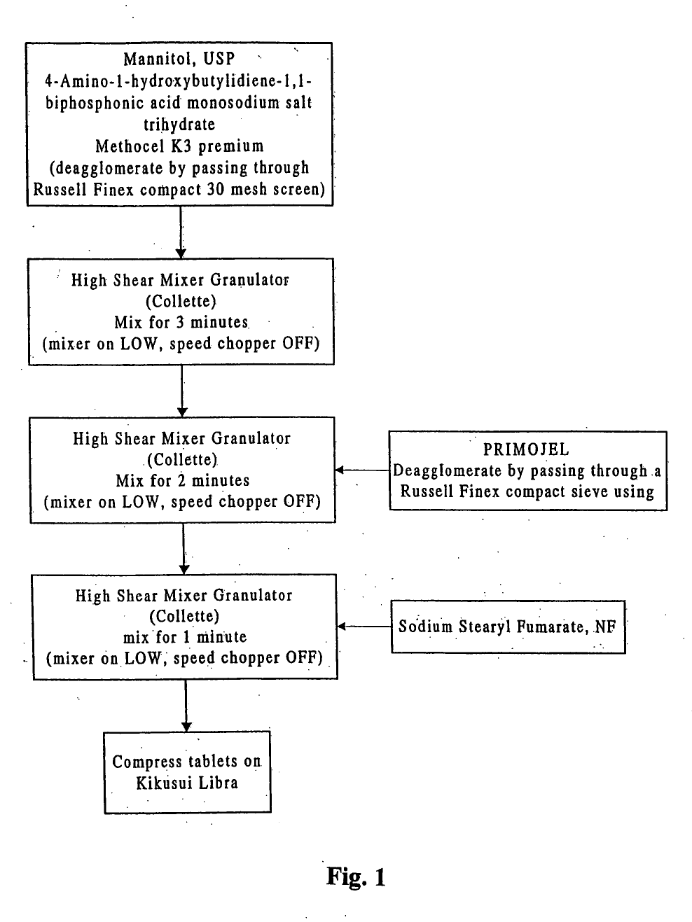

FIG. 1 illustrates the above-described tablet-forming process using preferred commercially available components of the composition of the invention.

### Examples 4-5

### Preparation of Tablets of 4-Amino-1-hydroxybutylidene-1,1-bisphosphonic acid monosodium salt trihydrate

| | Example 4 | Example 5 |
|---|---|---|
| | 35 mg Potency | 70 mg Potency |
| | Per Tablet | Per Tablet |
| Ingredients | (mg) | (mg) |
| 4-amino-1- | 45.685 | 91.37 |
| hydroxybutylidene-1,1- | | |
| bisphosphonic acid | | |
| monosodium salt | | |
| trihydrate | | |
| Mannitol, USP | 56.5 | 113.0 |
| (Pearlitol SD 200) | | |
| Microcrystalline | 70.065 | 140.13 |
| Cellulose, NF | | |
| (Avicel PH-101) | | |
| Croscarmellose | 1.75 | 3.5 |
| Sodium, NF | | |
| (Ac-Di-Sol) | | |
| Magnesium Stearate, | 1.0 | 2.0 |
| NF | | |
| Tablet Weight | 175 | 350 |

The following procedure was followed for each of the above tablet formulations:

a. mannitol, 4-amino-1-hydroxybutylidene-1,1-bisphosphonic acid monosodium trihydrate and microcrystalline cellulose were deagglomerated by passing through a screen;

b. the deagglomerated components from step (a) were mixed in a high shear mixer granulator for three minutes;

c. deagglomerated croscarmellose sodium was added and the mixture subjected to the action of a high shear mixer granulator for two minutes;

d. deagglomerated magnesium sterate was added and the mixture subjected to the action of a high shear mixer granulator for one minute; and

e. the final mixture was compressed into tablets.

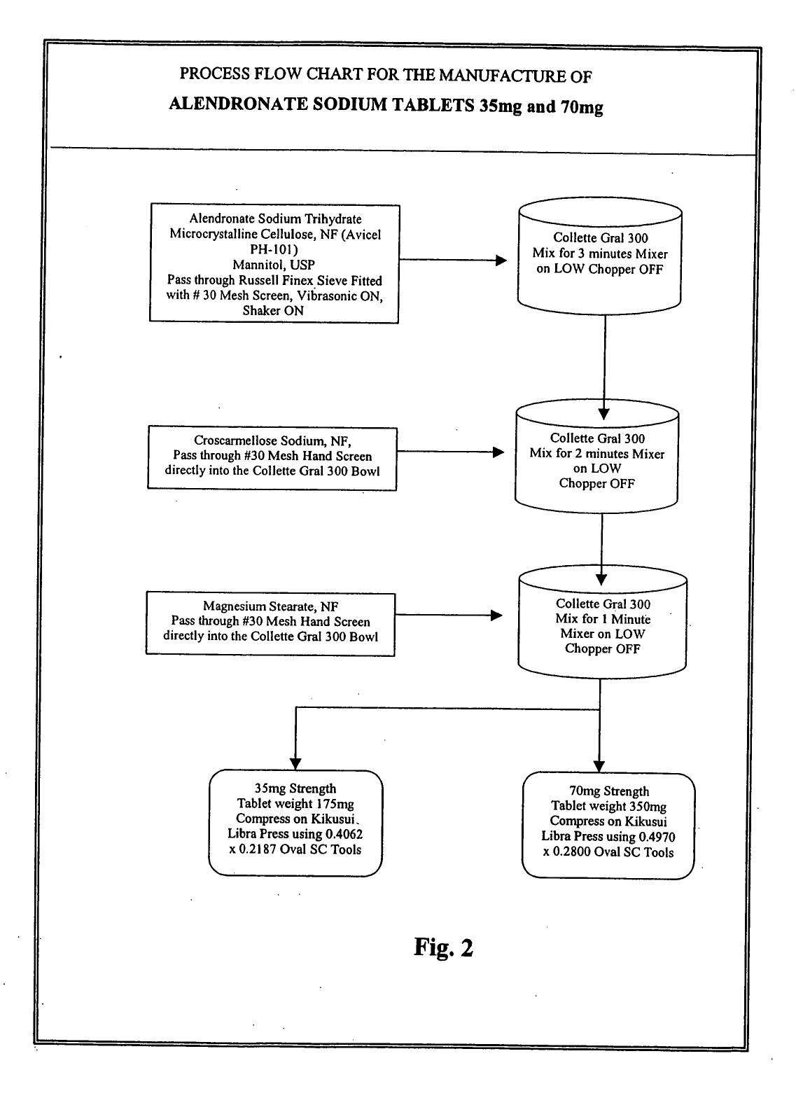

FIG. 2 illustrates the above-described tablet-forming process using preferred commercially available components of the composition of the invention.

### Example 6

### Comparative Dissolution Profile of 10 mg Tablets Prepared with a Mannitol Diluent and an Anhydrous Lactose Diluent

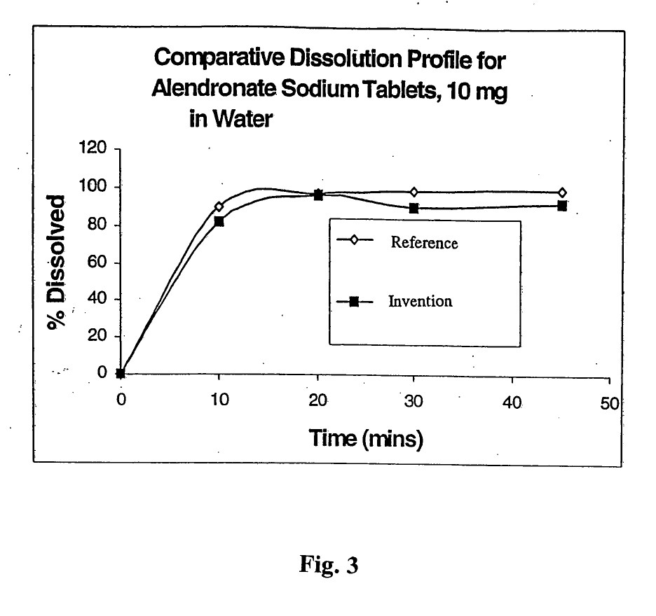

Tablet dissolution in 900 ml water for 10 mg tablets prepared with a non-reducing sugar diluent (Example 1, above) in accordance with the present invention was compared with that for commercially available 10 mg tablets prepared with anhydrous lactose (Fosamax® Tablets) using the paddle method, stirring at 50 rpm. The results are depicted graphically in FIG. 3. As shown in FIG. 3, both formulations were essentially completely dissolved after 10 minutes in water at 37° C. indicating that the substitution of mannitol for anhydrous lactose as a diluent in the formulation does not adversely effect the dissolution properties of the active bisphosphonic acid.

### Example 7

### Comparative Dissolution Profile of 40 mg Tablets Prepared with a Mannitol Diluent and an Anhydrous Lactose Diluent

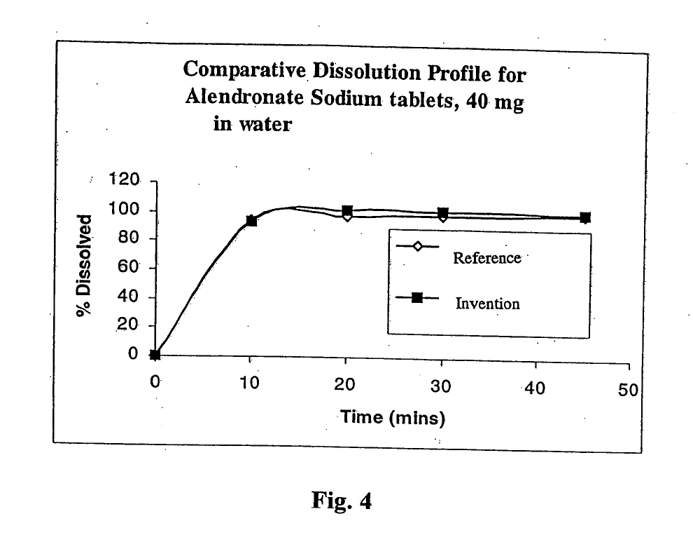

Tablet dissolution in 900 ml water for 40 mg tablets prepared with a non-reducing sugar diluent (Example 3, above) in accordance with the present invention was compared with that for commercially available tablets prepared with anhydrous lactose (Fosamax® Tablets) using the paddle method, stirred at 50 rpm. The results are depicted graphically in FIG. 4. As shown in FIG. 4, both formulations were essentially completely dissolved after 10 minutes in water at 37° C. indicating that the substitution of mannitol for anhydrous lactose as a diluent in the formulation does not adversely effect the dissolution properties of the active bisphosphonic acid.

### Example 8

### Comparative Dissolution Profile of 35 mg Tablets Prepared with a Mannitol Diluent and an Anhydrous Lactose Diluent

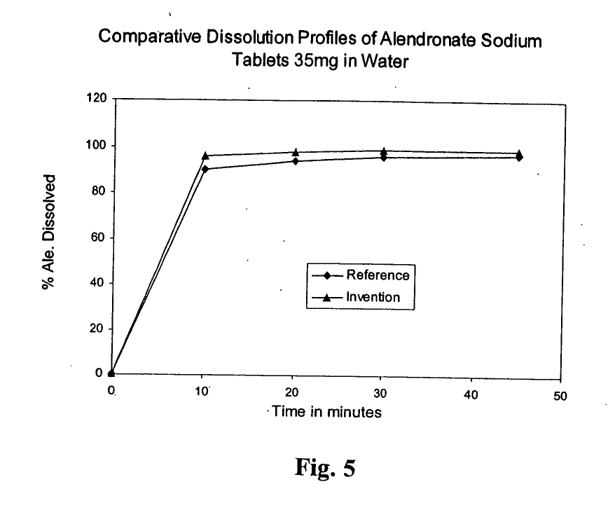

Tablet dissolution in 900 ml water for 35 mg tablets prepared with a non-reducing sugar diluent (Example 4, above) in accordance with the present invention was compared with that for commercially available 35 mg tablets prepared with anhydrous lactose (Fosamax® Tablets) using the paddle method at 50 rpm. The results are depicted graphically in FIG. 5. As shown in FIG. 5, both formulations were essentially completely dissolved after 10 minutes in water at 37° C. indicating that the substitution of mannitol for anhydrous lactose as a diluent in the formulation does not adversely effect the dissolution properties of the active bisphosphonic acid.

### Example 9

### Comparative Dissolution Profile of 70 mg Tablets Prepared with a Mannitol Diluent and an Anhydrous Lactose Diluent

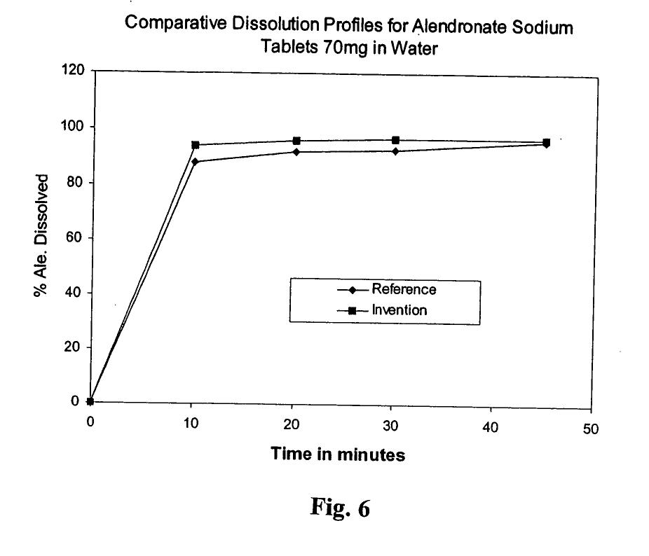

Tablet dissolution in 900 ml water for 70 mg tablets prepared with a non-reducing sugar diluent (Example 5, above) in accordance with the present invention was compared with that for commercially available 70 mg tablets prepared with anhydrous lactose (Fosamax® Tablets) using the paddle method at 50 rpm. The results are depicted graphically in FIG. 6. As shown in FIG. 6, both formulations were essentially completely dissolved after 10 minutes in water at 37° C. indicating that the substitution of mannitol for anhydrous lactose as a diluent in the formulation does not adversely effect the dissolution properties of the active bisphosphonic acid.

The pharmaceutical compositions of the present invention are useful in the therapeutic or prophylactic treatment of disorders in calcium or phosphate metabolism and associated diseases. These diseases include: osteoporosis (including estrogen defficiency, immobilization, glucocorticoid induced and senile), osteodystrophy, Paget's disease, myositis ossificans, Bechterew's disease, malignant hypercalcimia, metastatic bone disease, peridontal disease, cholelithiasis, nephrolithiasis, urolithiasis, urinary calculus, hardening of the arteries (sclerosis), arthritis, bursitis, neuritis and tetany.

Increased bone resorption can be accompanied by pathologically high calcium and phosphate concentrations in the plasma, which would be aleviated by use of the instant pharmaceutical compositons.

Having now fully described this invention, it will be understood to those of ordinary skill in the art that the same can be performed within a range of conditions, formulations, and other parameters without affecting the scope of the invention or any embodiment thereof All patents and publications cited herein are fully incorporated by reference herein in their entirety.

What is claimed is:

1. A pharmaceutical composition suitable for oral administration to a human, comprising:
from about 0.5% to about 60% by weight of a pharmaceutically acceptable salt of 4-amino-1-hydroxybutylidene-1,1-bisphosphonic acid;
(ii) from about 10% to about 95% by weight of a non-reducing sugar selected from the group consisting of mannitol, xylitol, sorbitol, inositol, sucrose and trehalose;
(iii) from about 2% to about 60% by weight of a binder selected from the group consisting of microcrystalline cellulose, hydroxypropyl cellulose, methyl cellulose, hydroxypropyl methyl cellulose and polyvinylpyrrolidone;
(iv) from about 0.5% to about 15% by weight of a disintegrant selected from the group consisting of starch, modified starch, croscarmellose sodium, crospovidone and sodium starch glycolate; and
(v) from about 0.1% to about 7% by weight of a lubricant selected from the group consisting of calcium stearate, magnesium stearate, stearic acid, talc, hydrogenated vegetable oil and sodium stearyl fumarate.

2. The pharmaceutical composition of claim 1, wherein said composition is in the form of a tablet.

3. The pharmaceutical composition according to claim 1, comprising:
(i) from about 0.5% to about 60% by weight of a pharmaceutically acceptable salt of 4-amino-1-hydroxybutylidene-1,1-bisphosphonic acid;
(ii) from about 30% to about 95% by weight of mannitol;
(iii) from about 2% to about 40% by weight of hydroxypropyl methyl cellulose;
(iv) from about 1% to about 15% by weight of sodium starch glycolate; and
(v) from about 0.25% to about 7% by weight of sodium stearyl fumarate.

4. The pharmaceutical composition according to claim 1, comprising:
(i) from about 10% to about 60% by weight of a pharmaceutically acceptable salt of 4-amino-1-hydroxybutylidene-1,1-bisphosphonic acid;
(ii) from about 10% to about 50% by weight of mannitol;
(iii) from about 20% to about 60% by weight of microcrystalline cellulose;
(iv) from about 0.5% to about 10% by weight of croscarmellose sodium; and
(v) from about 0.1% to about 3% by weight of magnesium stearate.

5. The pharmaceutical composition according to claim 1, wherein said a pharmaceutically acceptable salt of 4-amino-1-hydroxybutylidene-1,1-bisphosphonic acid is 4-amino-1-hydroxybutylidene-1,1-bisphosphonic acid monosodium salt trihydrate.

6. The pharmaceutical composition according to claim 1, wherein said a pharmaceutically acceptable salt of 4-amino-1-hydroxybutylidene-1,1-bisphosphonic acid is anhydrous 4-amino-1-hydroxybutylidene-1,1-bisphosphonic acid monosodium salt.

7. The pharmaceutical composition according to claim 1, comprising:
(i) from about 0.5% to about 50% by weight of a pharmaceutically acceptable salt of 4-amino-1-hydroxybutylidene-1,1-bisphosphonic acid;
(ii) from about 20% to about 90% by weight of a non-reducing sugar selected from the group consisting of mannitol, xylitol, sorbitol, inositol, sucrose and trehalose;
(iii) from about 5% to about 50% by weight of a binder selected from the group consisting of microcrystalline cellulose, hydroxypropyl cellulose, methyl cellulose, hydroxypropyl methyl cellulose and polyvinylpyrrolidone;
(iv) from about 0.5% to about 10% by weight of a disintegrant selected from the group consisting of starch, modified starch, croscarmellose sodium, crospovidone and sodium starch glycolate; and
(v) from about 0.25% to about 5% by weight of a lubricant selected from the group consisting of calcium stearate, magnesium stearate, stearic acid, talc, hydrogenated vegetable oil and sodium stearyl fumarate.

8. The pharmaceutical composition of claim 7, wherein said composition is in the form of a tablet.

9. The pharmaceutical composition according to claim 7, comprising:
(i) from about 0.5% to about 50% by weight of a pharmaceutically acceptable salt of 4-amino-1-hydroxybutylidene-1,1-bisphosphonic acid;
(ii) from about 40% to about 90% by weight of mannitol;
(iii) from about 5% to about 30% by weight of hydroxypropyl methyl cellulose;
(iv) from about 1% to about 10% by weight of sodium starch glycolate; and
(v) from about 0.5% to about 5% by weight of sodium stearyl fumarate.

10. The pharmaceutical composition according to claim 7, comprising:
(i) from about 15% to about 40% by weight of a pharmaceutically acceptable salt of 4-amino-1-hydroxybutylidene-1,1-bisphosphonic acid;
(ii) from about 20% to about 40% by weight of mannitol;
(iii) from about 30% to about 50% by weight of microcrystalline cellulose;
(iv) from about 0.5% to about 5% by weight of croscarmellose sodium; and
(v) from about 0.25% to about 2% by weight of magnesium stearate.

11. The pharmaceutical composition according to claim 7, wherein said a pharmaceutically acceptable salt of 4-amino-1-hydroxybutylidene-1,1-bisphosphonic acid is 4-amino-1-hydroxybutylidene-1,1-bisphosphonic acid monosodium salt trihydrate.

12. The pharmaceutical composition according to claim 7, wherein said a pharmaceutically acceptable salt of 4-amino-1-hydroxybutylidene-1,1-bisphosphonic acid is anhydrous 4-amino-1-hydroxybutylidene-1,1-bisphosphonic acid monosodium salt.

13. The pharmaceutical composition according to claim 7, comprising:
(i) from about 1% to about 30% by weight of a pharmaceutically acceptable salt of 4-amino-1-hydroxybutylidene-1,1-bisphosphonic acid;
(ii) from about 30% to about 80% by weight of a non-reducing sugar selected from the group consisting of mannitol, xylitol, sorbitol, inositol, sucrose and trehalose;
(iii) from about 10% to about 45% by weight of a binder selected from the group consisting of microcrystalline cellulose, hydroxypropyl cellulose, methyl cellulose, hydroxypropyl methyl cellulose and polyvinylpyrrolidone;
(iv) from about 0.5% to about 8% by weight of a disintegrant selected from the group consisting of starch, modified starch, croscarmellose sodium, crospovidone and sodium starch glycolate; and
(v) from about 0.5% to about 3% by weight of a lubricant selected from the group consisting of calcium stearate, magnesium stearate, stearic acid, talc, hydrogenated vegetable oil and sodium stearyl fumarate.

14. The pharmaceutical composition of claim 13, wherein said composition is in the form of a tablet.

15. The pharmaceutical composition according to claim 13, comprising:
(i) from about 1% to about 30% by weight of a pharmaceutically acceptable salt of 4-amino-1-hydroxybutylidene-1,1-bisphosphonic acid;
(ii) from about 50% to about 80% by weight of mannitol;
(iii) from about 10% to about 20% by weight of hydroxypropyl methyl cellulose;
(iv) from about 2% to about 8% by weight of sodium starch glycolate; and
(v) from about 1% to about 3% by weight of sodium stearyl fumarate.

16. The pharmaceutical composition according to claim 13, comprising:
(i) from about 20% to about 30% by weight of a pharmaceutically acceptable salt of 4-amino-1-hydroxybutylidene-1,1-bisphosphonic acid;
(ii) from about 25% to about 35% by weight of mannitol;
(iii) from about 35% to about 45% by weight of microcrystalline cellulose;
(iv) from about 0.5% to about 1.5% by weight of croscarmellose sodium; and
(v) from about 0.5% to about 1% by weight of magnesium stearate.

17. The pharmaceutical composition according to claim 13, wherein said a pharmaceutically acceptable salt of 4-amino-1-hydroxybutylidene-1,1-bisphosphonic acid is 4-amino-1-hydroxybutylidene-1,1-bisphosphonic acid monosodium salt trihydrate.

18. The pharmaceutical composition according to claim 13, wherein said a pharmaceutically acceptable salt of 4-amino-1-hydroxybutylidene-1,1-bisphosphonic acid is anhydrous 4-amino-1-hydroxybutylidene-1,1-bisphosphonic acid monosodium salt.

19. The pharmaceutical composition according to claim 15, comprising:
(i) from about 3% to about 26% by weight of a pharmaceutically acceptable salt of 4-amino-1-hydroxybutylidene-1,1-bisphosphonic acid;
(ii) from about 53% to about 76% by weight of mannitol;
(iii) about 13% to about 15% by weight of hydroxypropyl methyl cellulose;
(iv) about 4% to about 6% by weight of sodium starch glycolate; and
(v) about 2% by weight of sodium stearyl fumarate.

20. The pharmaceutical composition of claim 19, wherein said composition is in the form of a tablet.

21. The pharmaceutical composition according to claim 19, wherein said a pharmaceutically acceptable salt of 4-amino-1-hydroxybutylidene-1,1-bisphosphonic acid is 4-amino-1-hydroxybutylidene-1,1-bisphosphonic acid monosodium salt trihydrate.

22. The pharmaceutical composition according to claim 19, wherein said a pharmaceutically acceptable salt of 4-amino-1-hydroxybutylidene-1,1-bisphosphonic acid is anhydrous 4-amino-1-hydroxybutylidene-1,1-bisphosphonic acid monosodium salt.

23. The pharmaceutical composition according to claim 16, comprising:
(i) about 26% by weight of a pharmaceutically acceptable salt of 4-amino-1-hydroxybutylidene-1,1-bisphosphonic acid;
(ii) about 32% by weight of mannitol;
(iii) about 40% by weight of microcrystalline cellulose;
(iv) about 1% by weight of croscarmellose sodium; and
(v) about 0.6% by weight of magnesium stearate.

24. The pharmaceutical composition of claim 23, wherein said composition is in the form of a tablet.

25. The pharmaceutical composition according to claim 23, wherein said a pharmaceutically acceptable salt of 4-amino-1-hydroxybutylidene-1,1-bisphosphonic acid is 4-amino-1-hydroxybutylidene-1,1-bisphosphonic acid monosodium salt trihydrate.

26. The pharmaceutical composition according to claim 23, wherein said a pharmaceutically acceptable salt of 4-amino-1-hydroxybutylidene-1,1-bisphosphonic acid is anhydrous 4-amino-1-hydroxybutylidene-1,1-bisphosphonic acid monosodium salt.

27. A process for preparing a pharmaceutical composition suitable for oral administration to a human comprising:
(i) forming a mixture of from about 0.5% to about 60% by weight of a pharmaceutically acceptable salt of 4-amino-1-hydroxybutylidene-1,1-bisphosphonic acid, from about 10% to about 95% by weight of a non-reducing sugar selected from the group consisting of mannitol, xylitol, sorbitol, inositol, sucrose and trehalose, from about 2% to about 60% by weight of a binder selected from the group consisting of microcrystalline cellulose, hydroxypropyl cellulose, methyl cellulose, hydroxypropyl methyl cellulose and polyvinylpyrrolidone; from about 0.5% to about 15% by weight of a disintegrant selected from the group consisting of starch, modified starch, croscarmellose sodium, crospovidone and sodium starch glycolate; and from about 0.1% to about 7% by weight of a lubricant selected from the group consisting of calcium stearate, magnesium stearate, stearic acid, talc, hydrogenated vegetable oil and sodium stearyl fumarate; and
(ii) compressing said mixture into a tablet.
